(19) United States
(12) Patent Application Publication
(10) Pub. No.: US 2023/0079115 A1
(43) Pub. Date: Mar. 16, 2023

(54) ENERGY AND HYDROGEN LOGISTICS

(71) Applicant: MAN Energy Solutions SE, Augsburg (DE)

(72) Inventors: Rolf BANK, Deggendorf (DE); Norbert ANGER, Linz (AT)

(21) Appl. No.: 17/939,088

(22) Filed: Sep. 7, 2022

(30) Foreign Application Priority Data
Sep. 10, 2021 (DE) 10 2021 123 556.1

(51) Int. Cl. C01B 3/34; C25B 1/04

(52) U.S. Cl. CPC C01B 3/34; C25B 1/04; C01B 2203/1241; C01B 2203/0233

(57) ABSTRACT

A method for transporting liquid methane includes generating electricity in plants; using the electricity to split water into hydrogen and oxygen; providing carbon dioxide; feeding the hydrogen and the carbon dioxide from step into a reactor system for producing methane, wherein this reactor system comprises a catalytic reactor cooled with boiling water; liquefying the methane so produced; transporting the liquefied methane to a place of consumption located far away; utilising the liquefied methane at the place of consumption subject to generating carbon dioxide;) separating this carbon dioxide. At the place of consumption the methane is subjected to a steam reformation for producing hydrogen, wherein carbon dioxide is generated. At least a part of the carbon dioxide generated during the steam reformation is transported back to the reactor system for producing methane.

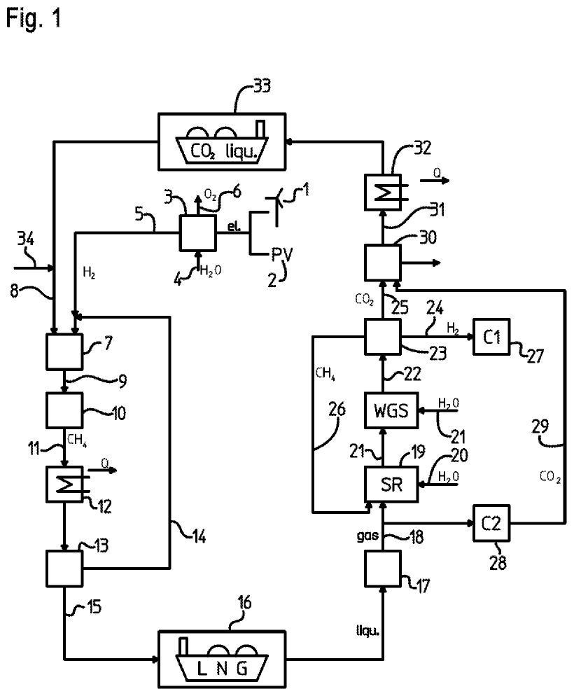

### BACKGROUND OF INVENTION

### 1. Field of the Invention

The disclosure relates to a method for transporting hydrogen in the form of liquid methane.

Using such methods, energy is converted into hydrogen and the same transformed into another chemical substance that causes lower transport costs than the transport of hydrogen. By way of this, energy from regions with excess energy, for example solar energy from the solar belt of the earth or wind energy from windy regions such as from the south of South America can be brought relatively cost-effectively to consumers located very far away, in particular on other continents. In the process it is known to tie the hydrogen generated through the conversion of the energy to a liquid organic hydrogen carrier (LOHC) through a hydrogenation reaction. Because of this, the hydrogen can be transported like a crude oil. At the destination, the hydrogen is liberated through a further chemical reaction (dehydrogenation of the LOHC) and is available.

### 2. Description of Related Art

In AU 2011101411 A4 a generic method is described, in particular a method for sequestering carbon dioxide by producing and exporting renewable liquid natural gas (LNG) including the following steps:

1. Generating hydrogen from renewable energy;

2. Feeding the hydrogen into a Sabatier reactor together with carbon dioxide from a carbon dioxide source of a local or overseas emitter in order to generate methane and water;

3. Transporting the methane to an Australian LNG plant from where it can be transported to LNG customers overseas by ships.

From US 2011/0064647 A1 a device and a method for storing and for transporting hydrogen using carbon dioxide as storage medium is known. An electrolyser uses energy from renewable sources for providing hydrogen by dissociation from water. A reactor forms a product, preferentially methane, through reaction of hydrogen and carbon dioxide. The product is transported to a place of consumption or the place of storage. A storage device can be used in order to store captured carbon dioxide developing during the usage of the product. This captured carbon dioxide is transported back to the reactor site in order to react there with the hydrogen provided from a hydrogen source. Thus, a carbon dioxide cycle is utilised in order to efficiently transport and store hydrogen.

DE 2940334 A1 discloses a method for methanizing a synthetic gas substantially containing hydrogen and oxides of the carbon by catalytic conversion at elevated temperatures and pressures. The methanization is conducted under excess hydrogen wherein the excess hydrogen is separated from the product flow and returned into the methanization.

### SUMMARY OF THE INVENTION

One aspect of the present invention is a method of the type mentioned at the outset so that the hydrogen quantity available at the place of consumption is increased.

According to one aspect of the invention it is possible that on the one hand a large hydrogen quantity (25% hydrogen compared with 6.3% with the LOHC concept) is transported in the carrier material and additionally, through a steam reformation of the methane and a possibly following water-gas shift reaction, the available quantity of hydrogen is doubled at the place of consumption. The CO₂ being generated can be separated and transported back so that the CO₂ is conducted in the cycle. By combining processes that are known per se in the order of
methanization and methane liquefaction at the energy-rich location on the one hand,
then transport (LNG transport, e.g. LNG tanker when transported by sea) and
steam reformation including a possible water-gas shift reaction at the place of consumption of the hydrogen on the other hand,
   0.5 kg of hydrogen is available per kilogram of transported carrier material (i.e. 50%) at the place of consumption.

Here, the invention is based on the realisation that comparing the different transport options by a hydrogen carrier (LOHC, ammonium, methanol, or LNG) the hydrogen portion with LNG is highest with 25% by mass. When the LNG is produced based on current, this is also called “eLNG”. In the case of LOHC, the hydrogen portion is 6.2% by weight, in the case of ammonia 17.6% by weight and in the case of methanol 12.5% by weight. The energy density that is transported by means of eLNG from the energy-rich region is highest with 15.5 kWh/kg (eLNG). LOHC supplies 2.1 kWh/kg (LOHC) via the combustion of the transported hydrogen, ammonia has an energy density of 6.25 kWh/kg (ammonia) and methanol with 6.3 kWh/kg (methanol) is at a comparable level to ammonia. This shows that in each case the transport of eLNG is most effective regardless of whether the transport of energy, hydrogen or both is the priority.

Thus, a process according to one aspect of the invention transforms the starting material “hydrogen” via the intermediate energy carrier “methane” back again into the starting material. In the process, this intermediate energy carrier takes up less volume and requires less expenditure for cooling and for compressing than other intermediate energy carriers. Thus, a larger quantity of energy at a higher temperature can be transported with the same transport volume.

Further, a process according to the invention can be utilised flexibly. Alternatively, it makes possible utilising the hydrogen transported with the help of the intermediate energy carrier or the energy carrier itself. Viewed as a whole, CO₂ is processed in a largely closed cycle.

For all individual processes of the overall method, commercially mature facilities are employed so that the method according to the invention can be implemented without delay.

It is questionable whether the transport of liquid hydrogen because of the problems of energy losses and of the safety expenditure will establish itself on a large technical scale. The process according to the invention overcomes these problems.

Preferably, the method includes a step, according to which the reaction gas generated through steam reformation is liquefied by cooling and during the cooling-down process the liquefied carbon dioxide separated from the gaseous hydrogen, and the separated carbon dioxide in the step c1) transported back by a CO₂ transport device. Using these measures, the hydrogen is effectively and cost-effectively separated from the carbon dioxide and the now liquefied carbon dioxide can now be likewise cost-effectively supplied again to the reactor system for producing methane.

Advantageously, a largely closed CO2 cycle is formed through the steps d) to f), g1), h) and c). CO₂ emissions are thus minimised.

In a favourable configuration of one aspect of the invention, the methanization is operated with an excess of hydrogen with respect to the conversion of the carbon dioxide of under 10% by volume, however with at least 0.3% by volume. The method is preferably operated at an operating pressure of at least 20 bar at the reactor input. Here, the excess hydrogen is preferably more than 1.0% by volume and particularly preferably more than 1.5% by volume. By operating the methanization with excess H2 (ratio of H₂: CO₂>4 in the reactor inflow), the CO₂ turnover is maximised. Only small to negligible quantities of CO₂, which precipitate on the cooling surfaces of the liquefaction plant as solid are present in the product gas and have to be removed from time to time by melting.

In an advantageous configuration of one aspect of the invention, the excess hydrogen is separated from the liquid methane in the gas phase and this excess hydrogen is returned again to the reactor system. By returning the excess H₂ into the inlet of the methanization reactor, no H₂ is lost.

Preferentially, the electricity generated is used for operating an electrolysis plant. A large technical implementation of the splitting of water is thus easily possible.

In a favourable further development of one aspect of the invention, step c) includes the collecting of carbon dioxide from an emission source, in particular from a methane-operated power plant, a biomass energy plant or an industrial plant emitting carbon dioxide. By way of this it is ensured that the carbon dioxide is provided in an environmentally friendly manner

### BRIEF DESCRIPTION OF DRAWINGS

The invention is additionally explained in more detail exemplarily by way of the only FIGURE:

The FIGURE shows an exemplarily method for transporting hydrogen in the form of liquid methane.

### DETAILED DESCRIPTION OF THE PRESENTLY PREFERRED EMBODIMENTS

The starting point of the method is formed by the generation of electric energy by way of renewable energy sources, for example from wind energy 1 or from solar energy 2. This electric energy is utilised—for example in an electrolyser 3—for splitting water 4 into hydrogen 5 and oxygen 6. In the simplest case, the oxygen 6 is released into the atmosphere. However, it can also be advantageously utilised in industrial processes or for supporting the biological wastewater purification in treatment plants. The generated hydrogen 5 is merged in a mixer 7 with a CO₂ flow 8 to form a reaction gas 9. Here, the mixing ratio is preferentially adjusted so that it contains hydrogen at a super-stoichiometric ratio. In a methanization reactor 10, the reaction gas 9 is converted into methane 11.

The converted reaction gas is cooled in a cooler 12 and liquefied. Condensable reaction products such as water are separated in a separator 13 and non-converted reaction gas components are again returned via a return line 14, preferably via the mixer 7, to the inlet into the methanization reactor 10. The methane 15 thus liquefied, also referred to as LNG (liquid natural gas) is fed into a specially equipped transport, in the case of a sea transport into a specially equipped tanker 16, and transported with the same to a place of consumption geographically further distant.

On arrival there, it is buffer-stored if applicable in tanks. In an evaporator 17, it is again transformed into gaseous methane 18. The methane 18 is converted in a steam reformer (SR) 19 with steam 20 to form synthetic gas 21 consisting of carbon dioxide and hydrogen. In a preferred embodiment, the reaction gas, by supplying further steam (WGS, water-gas shift) 21 the reaction gas is finally converted to form a product gas 22. In a separator 23, the same is, cooled, separated into its main constituent hydrogen (H₂, gaseous) 24 and carbon dioxide (CO₂, liquid) 25. Non-converted methane 26 is again returned into the steam reformer 19. The generated hydrogen 24 is fed to a hydrogen user (C1) 27. Another utilisation path of the gaseous methane 18 flowing out of the evaporator 17 is the conventional direct utilisation by a consumer (C2) 28. This can be for example a gas power plant, an industrial operation or a heating plant. Generally, this utilisation consists of combustion processes subject to forming CO₂, 29. Preferably, the latter is separated from the exhaust gas and in the same manner as the CO₂ 25 generated in the preferred embodiment supplied to a cleaning stage 30. The CO₂ 31 cleaned in this manner is liquefied in a cooler 32 and in a transport 33 that is identical or similar to the transport 16 returned to the mixer 7 of the methanization plant. It is likewise possible that the transport means 16 is used as transport means 33 for the CO₂.

The CO₂ 31 from the consumption processes is largely reclaimed and conducted in the cycle. The term “largely” is to mean that as much as possible CO₂ is reclaimed. However, as with any eclamation process there are losses that cannot be entirely avoided for technical and economic reasons. The losses created have to be offset through other CO₂ sources. The CO2 supplementation flow 34 originating from there is fed back to the mixer 7 together with the CO₂ flow 8.

The conveying devices required for the various part processes are known to the person skilled in the art. There is therefore no detailed representation here. Further, modifications of individual method steps such as for example the switching of cleaning and cooling stages are within the scope of the invention. The plant-internal heat utilisation of heat generating and heat utilising processes is likewise known to the person skilled in the art and is not discussed further here.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1. A method for transporting hydrogen as liquid methane, comprising:
a) generating electricity;
b) using the electricity generated in order to split water into hydrogen and oxygen;
c) providing carbon dioxide, ;
d) feeding the hydrogen from b) and the carbon dioxide from c) into a reactor system configured to produce methane, wherein the reactor system comprises a catalytic reactor cooled with boiling water;
e) liquefying the methane;
f) transporting the liquefied methane to a place of consumption located a distance away;
g) utilising the liquefied methane at the place of consumption subject to generating carbon dioxide, wherein the methane is subjected to a steam reformation to produce gaseous hydrogen, wherein carbon dioxide is generated; and
h) separating the carbon dioxide;
wherein c) includes:
a return transport of carbon dioxide from h); and
c1) at least a part of the carbon dioxide generated during the steam reformation is transported back to the reactor system for producing methane.

2. The method according to claim 1,
wherein h) includes the following:
liquefying reaction gas generated through the steam reformation by cooling and during the cooling, the carbon dioxide, which is liquefied, is separated from the gaseous hydrogen; and
wherein in c1) the separated carbon dioxide is transported back into the reactor system according to d) by a CO₂ transport.

3. The method according to claim 1, wherein through d) to f), g1), h) and c1) a largely closed CO₂ cycle is formed.

4. The method according to claim 1, wherein in c) methanization is operated with an excess of hydrogen with respect to conversion of the carbon dioxide of under 10% by volume, with at least 0.3% by volume.

5. The method according to claim 4, wherein the excess of hydrogen amounts to more than 1.0% by volume.

6. The method according to claim 5, wherein the excess of hydrogen amounts to more than 1.5% by volume.

7. The method according to claim 1, wherein
in e) excess hydrogen is separated from the liquid methane in a gas phase, and
in d) the excess hydrogen is returned to the reactor system.

8. The method according to claim 1, wherein in b) the electricity generated in a) is used for operating an electrolysis plant.

9. The method according to claim 1, wherein c) includes:
collecting of carbon dioxide from an emission source.

10. The method according to claim 9, wherein the emission source is a power plant operated with methane, a biomass energy plant, or an industrial plant emitting carbon dioxide.

11. The method according to claim 1, wherein the electricity is generated in a plant that utilises renewable energies.

12. The method according to claim 11, wherein the renewable energies comprise wind, solar, biomass, or geo-thermal.
